(19) United States
(12) Patent Application Publication
(10) Pub. No.: US 2016/0158061 A1
(43) Pub. Date: Jun. 9, 2016

(54) TECHNIQUE FOR SETTING ENERGY-RELATED LASER-PULSE PARAMETERS

(71) Applicant: WAVELIGHT GMBH, Erlangen (DE)

(72) Inventors: Matthias Foesel, Memmelsdorf (DE); Christof Donitzky, Eckental/Eschenau (DE); Klaus Vogler, Eckental/Eschenau (DE); Olaf Kittelmann, Berlin (DE)

(21) Appl. No.: 14/787,240

(22) PCT Filed: May 22, 2014
(86) PCT No.: PCT/EP2014/060565

§ 371 Date: Oct. 26, 2015

(51) Int. Cl. A61F 9/008

(52) U.S. Cl. CPC A61F 9/0084

(57) ABSTRACT

A method for energy setting of pulsed, focused laser radiation is provided. In the method, a relationship between a threshold pulse energy required for causing irreversible damage in a material and a pulse duration is established. The relationship allows for obtaining a threshold pulse energy for each of a plurality of pulse durations, including one or more pulse durations in a range between 200 fs and smaller. The relationship defines a decreasing threshold pulse energy for a decreasing pulse duration in the range between 200 fs and smaller. For a given pulse duration in the range between 200 fs and smaller, an associated threshold pulse energy is determined based on the established relationship. The pulse energy of the laser radiation is set based on the determined associated threshold pulse energy.

### TECHNICAL FIELD

The present disclosure relates to setting laser-pulse parameters. It relates in particular to techniques for setting energy-related laser-pulse parameters.

### BACKGROUND

Especially in industrial and medical environments, pulsed focused laser radiation has become an important means for material processing. In typical applications of pulsed laser radiation, electromagnetic and/or thermal effects of absorbed laser radiation are used to locally change or disrupt a target material in an irradiated region thereof in order to create a cut in, or ablate matter from, the target. Focusing the incident radiation allows for an increased local intensity of the radiation and for a spatially closer confined zone of interaction with the target material. In addition, the use of pulsed radiation instead of continuous radiation reduces the effect of heat accumulation in the treated object.

In medical laser applications, e.g., in laser-assisted eye surgery including, but not limited to, LASIK (Laser in-situ Keratomileusis), Keratoplasty, refractive lenticule extraction, etc., and also in other types of material processing using laser radiation, a sharply defined scope of the laser treatment and a low total transfer of energy into the treated material are usually desired. For that purpose, the use of ultra-short laser pulses having a pulse width in the range below one picosecond has been suggested. Conventionally used pulse durations may be anywhere between 250 fs and 800 fs, for example. At the same time, it is attempted to set the energy of each single pulse as small as possible, i.e., close to a threshold energy for achieving disruption or any other desired effect in the target.

When adjusting required pulse energies to different pulse durations for reaching a desired effect, it has been observed that, although shorter pulses may have a higher threshold power, the product of threshold power and pulse duration, i.e., the threshold pulse energy, often decreases for shorter pulse durations. However, it is known in this regard that for various applications there exist characteristic pulse widths below which the threshold pulse energy can be observed to increase again. In that case, given that a minimal transfer of energy is desired, ideal pulse lengths could be determined, which may only vary for different applications and different beam or target parameters.

Ideal laser pulse characteristics depend substantially on the treated material and the intended effect of a laser treatment. For example, in many practical applications a modification of the pulse length may become desirable in connection with a change of the process. Problems may arise when the settings of energy-related pulse parameters that were tuned to fit a particular pulse length become suboptimal in terms of a minimal energy transfer when the pulse length is changed. At the same time, a user of a laser apparatus may not be able to easily identify, e.g., during processing, the ideal pulse energy for a chosen pulse length and thus may unduly stress the target by exposing it to radiation high above the required energy level.

This has again particular disadvantages in surgery, especially in laser eye surgery, when cuts under a surface of the eye are performed. For, in such cases excessive pulse energy often leads to undesirably large vapor bubbles within the eye tissue in consequence of evaporated eye tissue; the size of the bubbles may be substantially larger than the focus diameter of the laser beam itself. Such bubbles stress the surrounding eye tissue and they change the optical properties of the operation zone such that the laser process itself, or related optical techniques, are negatively affected. Moreover, if in the described scenario a series of pulses is directed in close proximity to one another, the produced bubbles can connect to even larger cells and thus enhance their negative effects. While several techniques for removing such gas volumes are known, it would be beneficial if, for a range of different applications, their occurrence could be held at a minimum. This, however, requires an adaptation of laser parameters with a change of the application.

A technique for facilitated setting of laser pulse parameters is therefore desirable.

### SUMMARY

According to a first aspect a method for energy setting of pulsed, focused laser radiation is described. The method comprises the steps of establishing a relationship between a threshold pulse energy required for causing irreversible damage in a material and a pulse duration, the relationship allowing to obtain a threshold pulse energy for each of a plurality of pulse durations, the plurality of pulse durations including one or more pulse durations in a range between 200 fs and smaller; for a given pulse duration in the range between 200 fs and smaller, determining an associated threshold pulse energy based on the established relationship; and setting the pulse energy of the laser radiation based on the determined associated threshold pulse energy, wherein the relationship defines a decreasing threshold pulse energy for a decreasing pulse duration in the range between 200 fs and smaller.

The relationship may represent a decrease of the threshold pulse energy substantially as a function of the cubic root of the pulse duration. In certain embodiments, the function is a linear function of the cubic root of the pulse duration. In addition or as an alternative, the relationship may define the threshold pulse energy as a value of at most 0.35 μJ, e.g., at most 0.30 μJ or at most 0.25 μJ or at most 0.20 μJ or at most 0.15 μJ, for a pulse duration of 300 fs or smaller. In addition or as an alternative, the relationship may define the threshold pulse energy as a value in the range from 0.15 μJ to 0.30 μJ, e.g., in the range from 0.15 μJ to 0.20 μJ or from 0.20 μJ to 0.25 μJ or from 0.25 μJ to 0.30 μJ or from 0.20 μJ to 0.30 μJ, for a pulse duration of 200 fs. In addition or as an alternative, the relationship may define the threshold pulse energy as a value in the range from 0.05 μJ to 0.10 μJ, e.g., in the range from 0.05 μJ to 0.08 μJ or from 0.08 μJ to 0.10 μJ, for a pulse duration of 10 fs.

The establishing step may include the steps of irradiating, for each of a plurality of reference pulse durations above 200 fs, an object with a series of pulses of the laser radiation to create a damage site for each pulse of the series, wherein the pulse energy is set differently for each pulse of the series, determining a size of each damage site, determining a reference threshold pulse energy for each of the plurality of reference pulse durations based on the determined sizes of the damage sites created at the respective reference pulse duration, and determining the relationship based on the determined reference threshold pulse energies. The object may be a non-biological material or a post mortem biological material.

Each reference threshold pulse energy may be determined based on an extrapolation to zero size of the determined sizes of the damage sites created at the respective reference pulse duration. The sizes may be determined, for example, based on a diameter, an area or a volume of each damage site. The extrapolation may be based, for example, on a linear, an exponential, or a polynomial fit or any combination thereof applied to the determined sizes.

In addition or as an alternative, determining the relationship may include determining a linear approximation of the threshold pulse energy in dependence on the pulse duration.

The relationship may be established for a focus diameter of the laser radiation of no more than 10 μm or 7 μm or 5 μm, wherein the focus diameter represents the diameter of a pulse portion containing 86% of the energy of a pulse of the radiation.

The damage may include a photodisruption caused by a laser-induced optical breakdown of the material.

The method may include the step of directing the laser radiation having the set pulse energy at a non-biological material or a biological material to create an incision in the material. The material may be human eye tissue.

The relationship may be established between the pulse duration and, in place of the threshold pulse energy, a threshold pulse fluence required for causing irreversible damage in the material, wherein the relationship defines a decreasing threshold pulse fluence for a decreasing pulse duration in the range between 200 fs and smaller, and wherein an associated threshold pulse fluence is determined in place of the associated threshold pulse energy and the pulse fluence of the laser radiation is set based on the determined associated threshold pulse fluence.

If the relationship is established between the pulse duration and a threshold pulse fluence, the relationship may further define the threshold pulse fluence as a value of at most 1.80 Jcm⁻², e.g., at most 1.50 Jcm⁻² or at most 1.30 Jcm⁻² or at most 1.10 Jcm⁻² or at most 0.90 Jcm⁻² or at most 0.70 Jcm⁻² or at most 0.50 Jcm⁻², for a pulse duration of 300 fs or smaller. In addition or as an alternative, the relationship may define the threshold pulse fluence as a value in the range from 0.80 Jcm⁻² to 1.50 Jcm⁻², e.g., in the range from 0.80 Jcm⁻² to 0.95 Jcm⁻² or from 0.95 Jcm⁻² to 1.05 Jcm⁻² or from 1.05 Jcm⁻² to 1.30 Jcm⁻² or from 1.30 Jcm⁻² to 1.50 Jcm⁻², for a pulse duration of 200 fs. In addition or as an alternative, the relationship may define the threshold pulse fluence as a value in the range from 0.20 Jcm⁻² to 0.50 Jcm⁻², e.g. in the range from 0.20 Jcm⁻² to 0.35 Jcm⁻² or from 0.35 Jcm⁻² to 0.50 Jcm⁻², for a pulse duration of 10 fs.

According to a second aspect a laser apparatus is described, the laser apparatus comprising a source of a beam of ultrashort-pulsed laser radiation, a set of components for guiding and shaping the beam in time and space, a control unit storing data representative of a relationship between a threshold pulse energy required for causing irreversible damage in a material and a pulse duration, the relationship allowing to obtain a threshold pulse energy for each of a plurality of pulse durations, the plurality of pulse durations including one or more pulse durations in a range between 200 fs and smaller, wherein the relationship defines a decreasing threshold pulse energy for a decreasing pulse duration in the range between 200 fs and smaller, wherein the control unit is configured to determine for a given pulse duration in the range between 200 fs and smaller an associated threshold pulse energy based on the stored data and to determine a target pulse energy for the beam based on the determined associated threshold pulse energy.

The control unit may be configured to output a visual representation of the determined target pulse energy on an output device. The output device may be a remote device or may be integral with the laser apparatus. In addition or as an alternative, the control unit may be configured to set the determined target pulse energy for the beam automatically.

The relationship may represent a decrease of the threshold pulse energy substantially as a function of the cubic root of the pulse duration. In addition or as an alternative, the relationship may define the threshold pulse energy as a value of at most 0.35 μJ, e.g., at most 0.30 μJ or at most 0.25 μJ or at most 0.20 μJ or at most 0.15 μJ, for a pulse duration of 300 fs or smaller. In addition or as an alternative, the relationship may define the threshold pulse energy as a value in the range from 0.15 μJ to 0.25 μJ, e.g., in the range from 0.18 μJ to 0.22 μJ, for a pulse duration of 200 fs. In addition or as an alternative, the relationship may define the threshold pulse energy as a value in the range from 0.05 μJ to 0.10 μJ, e.g., in the range from 0.06 μJ to 0.08 μJ, for a pulse duration of 10 fs.

The beam may be a Gaussian beam having an M² parameter of no more than 1.15 or 1.1.

Further details, objects and advantages of the invention become apparent from the following description and drawings.

### BRIEF DESCRIPTION OF THE DRAWINGS

The invention is illustrated with reference to the following diagrams.

FIG. 1 is a schematic representation of an embodiment of a method for determining threshold pulse energies for individual pulse durations according to the present invention;

FIG. 2 is a schematic representation of an embodiment of a method for determining threshold pulse energies for a range of pulse durations according to the present invention;

FIG. 3 is a flowchart of an embodiment of a method for energy setting of pulsed, focused laser radiation according to the present invention;

FIG. 4 is a flowchart of an alternative embodiment of a method for energy setting of pulsed, focused laser radiation according to the present invention; and

FIG. 5 is a schematic representation of an embodiment of a laser apparatus according to the present invention.

### DETAILED DESCRIPTION

FIG. 1 illustrates schematically an embodiment of a method for determining, for particular pulse durations and for an arbitrary target material, a threshold pulse energy required to cause irreversible damage in the target material. In the example of FIG. 1, the pulse durations T_{L} are chosen as 300 fs, 400 fs and 500 fs, but the described method can also be applied to any other set of pulse lengths.

As shown in the diagram of FIG. 1, for any chosen pulse length a damage of finite size, D_{Damage}, will occur in the target material as soon as a pulse length-dependent threshold energy, Eₜₕ, is reached or exceeded. The diagram further shows that, for any given pulse length T_{L}, the size D_{Damage} of a damage site caused in the target material will increase with the pulse energy. From a comparison of the three curves it can be seen that a similar extent of damage as caused by a pulse of 500 fs and with energy E₂ can also be achieved with less energy, E₁, if that energy is concentrated in a shorter pulse of 300 fs. This agrees with the general assumption that, for similar effects, the use of shorter pulses allows for a smaller amount of energy transferred.

Since at the respective threshold pulse energies, E_{th,300}, E_{th,400}, E_{th,500}, the damage that is caused by a single pulse would be invisibly small, i.e., D_{Damage}=0 and/or consist only in thermal changes in the material, the extent of the damage is determined for higher energies, E₁, E₂, where for a range of pulse durations the sizes of damage sites can be conveniently measured. As indicated by the dashed lines in FIG. 1, extrapolating that dependency for each pulse duration to a damage size of zero will subsequently yield values for the corresponding threshold energies.

Although the curves in FIG. 1 suggest a linear dependency of the damage size on the pulse energy, the dependency may often be better described by a non-linear relation. The exact relationship depends, for example, on the quantity that is chosen to describe the damage size, either by a length, an area or a volume. Moreover, whereas in the described example only single-pulse effects have been considered, the method may equally involve a detection of damage sizes for varying numbers of pulses applied at the same location (i.e. pulse bursts).

While conventional methods for determining threshold energies often rely on secondary effects that occur in connection with laser-induced optical breakdown, e.g., a rapid increase of plasma emission, the present method measures directly the intended effect of irreversible damage in the target material. In that way, threshold energies could be determined, experimentally and for different target materials, which differ from the results gained by other methods. The experiments suggest in particular that irreversible disruption can be achieved at lower threshold energies than is generally assumed. This, however, does not exclude the possibility that also in the present method the damage in the target material is at least partly caused by laser-induced optical breakdown.

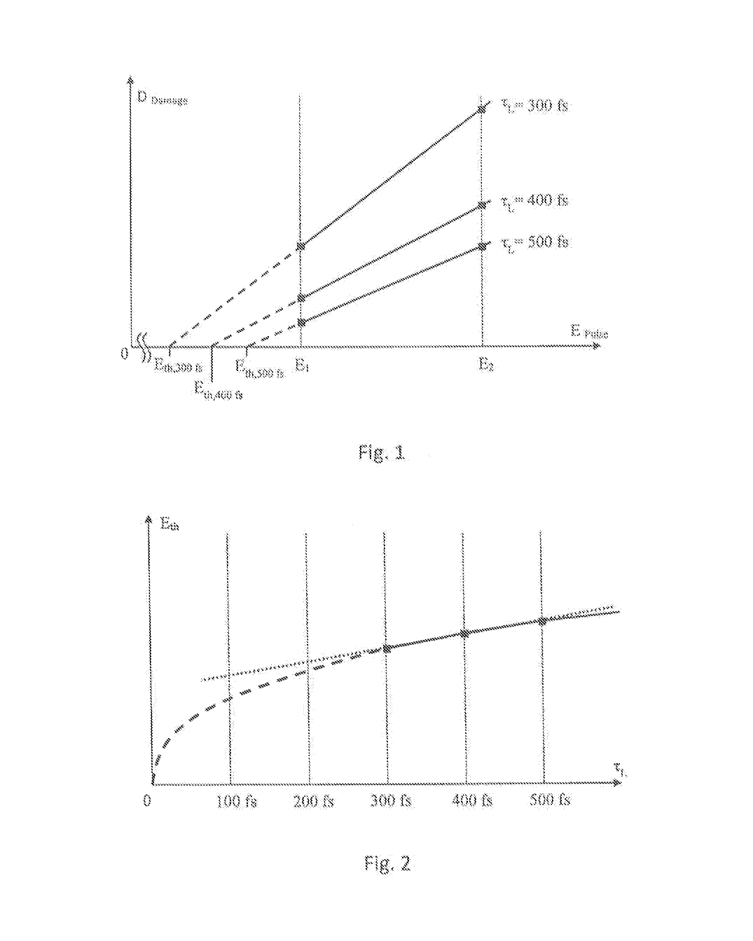

FIG. 2 illustrates, schematically and for arbitrary pulse and material characteristics, a method for determining a relationship between the damage threshold pulse energy and pulse durations in ranges below and above 200 fs. In the range above 200 fs, the different threshold energies, Eₜₕ, according to FIG. 1 have been plotted for the corresponding pulse lengths T_{L}=300 fs, 400 fs and 500 fs. As illustrated by the continuous curve in FIG. 2, interpolation allows one to establish the desired relationship in that range.

Based on the assumption that for ultrashort pulses disruption can be conceived as a mainly intensity-dependent process, which poses no lower boundary to the pulse length and, thus, to the threshold energy, it has been further assumed that the relationship expressed by the curve in FIG. 2 may start from the origin. In that case it is assumed that the threshold pulse energy is substantially a function of the cubic root of the pulse duration (E_{th(damage)}˜T^{1/3}). In connection with the measured data, and as illustrated by the dashed curve in FIG. 2, this allows an extrapolation of the curve beyond the measured range, also for pulse durations that are considerably shorter than 200 fs.

The resultant curve shows a continuous decrease of the threshold energy even towards shortest pulse lengths, and it is suitably described by a power function of the pulse length with an exponent smaller than 1. The curve thus implies that, if a low energy transfer into the target material is intended, pulse lengths can be reduced below the common usage, into the sub-200 fs-range, while the threshold pulse energy decreases steadily for decreasing pulse durations. Once the described relationship has been established, it may be used for setting the pulse energy for various pulse durations in the range between 200 fs and less.

In addition to the above, or in a simplified embodiment which suffices without the assumption that the curve passes through the origin, the relationship may at least partially be determined on grounds of a linear approximation based on the measured data. This variant is exemplarily illustrated by the dotted curve in FIG. 2. Furthermore, since the described relationship relies on the assumption of a predominant intensity-dependency, it can be advantageous in alternative embodiments of the described method to ignore the pulse energy in favor of other energy-related beam parameters, such as the fluence per pulse.

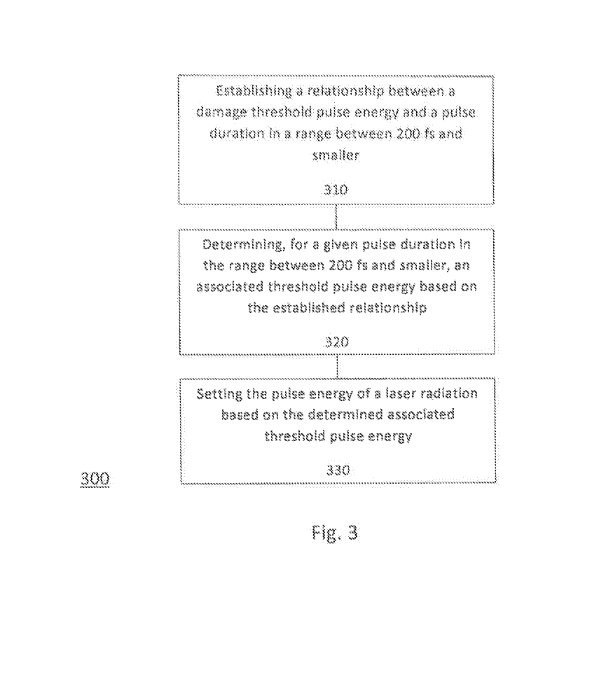

FIG. 3 is a flowchart of an exemplary embodiment of a method 300 for energy setting of pulsed, focused laser radiation according to the present invention. The method 300 may involve some or all of the above described procedures and results. In a first step 310, a relationship between a damage threshold pulse energy and a pulse duration in a range between 200 fs and less is established. This can be performed, for example, by the procedures described in connection with FIGS. 1 and 2. Based on that relationship, and for a given pulse duration in the range between 200 fs and less, an associated threshold pulse energy is determined, step 320. Subsequently, in step 330, the pulse energy of a laser radiation is set based on the determined associated threshold pulse energy.

The method 300 thus allows to easily adjust the energy of laser radiation to a changed pulse length. In this way it becomes possible, for example, to vary for particular processes a pulse length of a laser while always maintaining an optimized pulse energy. If the pulse energy is set at a value larger than the determined threshold energy, the set value may in certain embodiments be, e.g., in a range between 1.5 and 5 times or between 1.5 and 4 times or between 1.8 and 3.5 times or between 2 and 4 times the determined threshold energy. According to other embodiments, the pulse energy of a laser apparatus used for processing a material may be set at at least 1.3 times or at least 1.5 times or at least 1.8 times or at least 2.0 times the determined threshold energy. As for an upper limit, the set pulse energy may be no more than 5 times or no more than 4.5 times or no more than 4 times or no more than 3.5 times or no more than 3.0 or no more than 2.5 times the determined threshold energy. In certain embodiments, the pulse energy may be set by a predetermined absolute amount, e.g., 0.05 μJ, 0.10 μJ or 0.20 μJ or 0.30 μJ or 0.40 μJ, above the determined threshold energy. In any of such cases, information about the threshold energy for a range of pulse lengths provides relevant means for optimizing the pulse energy accordingly.

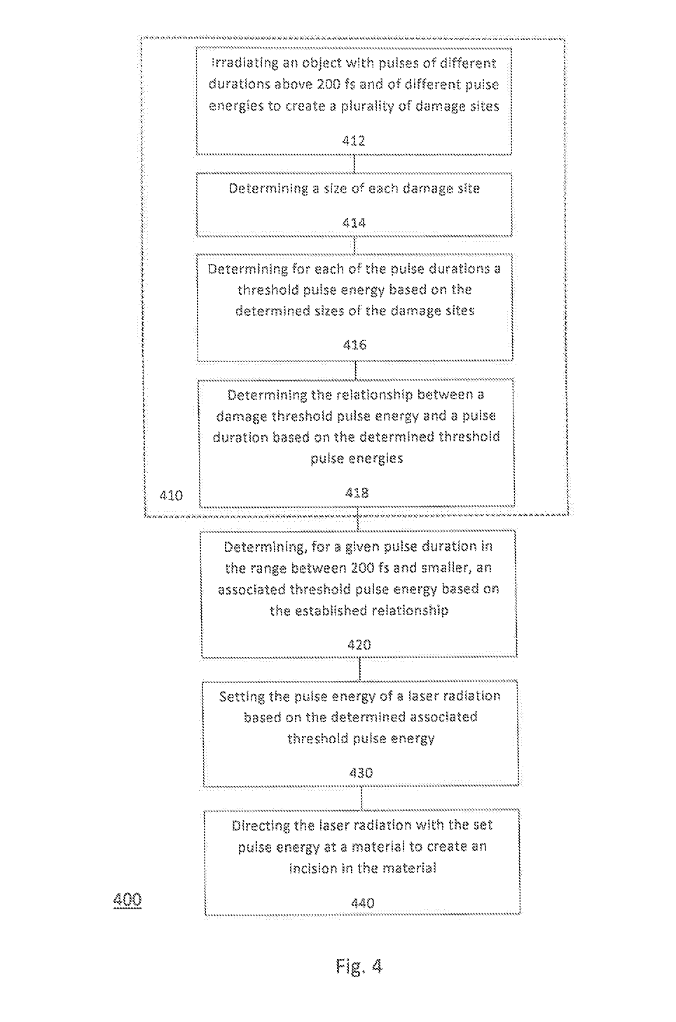

FIG. 4 shows a flowchart of an alternative embodiment of a method 400 for energy setting of pulsed, focused laser radiation according to the present invention. In the method 400 of FIG. 4, the step 410 for establishing a relationship between a damage threshold pulse energy and a pulse duration includes a plurality of sub-steps 412, 414, 416, 418. Analogous to the method 300 of FIG. 3, once that relationship has been established, it may be used to determine for a given pulse duration in the range between 200 fs and smaller an associated threshold pulse energy, step 420, and to set the pulse energy of a laser radiation based on the associated threshold pulse energy, step 430. Once the pulse energy has been set, the laser radiation can finally be directed at a target material to create an incision in the material, step 440.

In a first step 412 an object or sample of the material for which the described relationship is to be established is irradiated with different pulse energies and pulse durations above 200 fs such that measurable damage sites are produced in the object. The size of each damage site is then determined, step 414. Based on the determined sizes, a threshold pulse energy can be determined for each of the pulse durations, step 416. This may be performed by using any of the techniques described in connection with FIG. 1. Finally, based on the determined threshold energies, the relationship between a damage threshold pulse energy and a pulse duration is determined, step 418. This, again, can be done by using any of the techniques described in connection with FIG. 2.

An exact relationship between a damage threshold pulse energy and a pulse duration will depend also on numerous other conditions. These conditions include, most prominently, characteristics of the irradiated material and further beam parameters, such as the laser wavelength and the temporal and spatial profiles of the laser pulses. However, based on experimental data it turned out that for relevant applications the exponent of the power function that describes the sought relationship, as shown in FIG. 2, varies mainly between 0.3 and 0.36. It can therefore practically be approximated as a cubic root function of the pulse duration.

Moreover, the described method 300, 400 yields reliable results for different transparent non-biological and post mortem biological test materials such as Polymethylmethacrylat, PMMA, and animal eye tissue, and for beam characteristics in the most relevant ranges for established applications, e.g. when the diameter of the laser focus, i.e., the diameter of a beam cross-section that transmits ca. 86% of the pulse energy, is chosen smaller than 10 micrometers, e.g., smaller than 8 micrometers or smaller than 6 micrometers or smaller than 4 micrometers. For example, for pulse durations shorter than 300 fs, threshold energies between 0.05 and 0.35 micro-joules were determined. Within that range, the threshold energy for a pulse duration of 200 fs was determined to fall in a range from 0.15 to 0.30 micro-joules, and for a pulse duration of 10 fs, in a range from 0.05 to 0.1 micro-joules. It can therefore be expected, that the same characteristic cubic root-dependency of the threshold pulse energy on the pulse duration, and possibly also the same energy ranges, will apply in the case that the target material is human eye tissue.

As said before, alternative embodiments of the methods 300, 400 can be realized, in which other energy-related parameters, e.g., a fluence per pulse, instead of the pulse energy are considered. In such cases, too, the cubic-root dependency applies accordingly. For instance, with the same focus and material characteristics as in the previous example, and if applied to a threshold pulse fluence, the described method 300, 400 yields for pulse lengths less than 300 fs a threshold fluence between 0.2 and 1.80 Jcm⁻². More particular, the threshold fluence for a pulse duration of 200 fs was determined to fall in a range from 0.80 Jcm⁻²to 1.50 Jcm⁻², and for a pulse duration of 10 fs, in a range from 0.20 Jcm⁻² to 0.50 Jcm⁻².

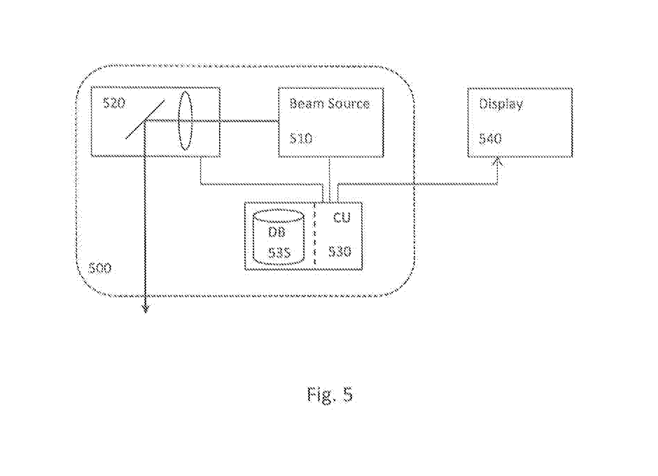

FIG. 5 shows an exemplary embodiment of a laser apparatus 500 according to the present invention. The laser apparatus 500 comprises a beam source 510, a set 520 of components for guiding and shaping the beam in time and space, and a control unit 530. The control unit 530 may comprise, or be connected to, a data base 535 such that the control unit 530 can access and process data that is stored by the data base 535. The laser apparatus 500 may further include a visual output device 540 and/or be adapted to output, by the control unit 530 and for visual representation, a signal to an external output device 540.

For simplicity, the beam source 510 and the set 520 of guiding and shaping components are shown as two distinct entities in FIG. 5. In alternative embodiments, however, the means for beam shaping and guiding may comprise a plurality of disjoint components in the laser apparatus 500; conversely, also the beam source 510 may comprise means for guiding and shaping a generated laser beam. It should therefore be understood that the beam source 510 together with the guiding and shaping means 520 define any technical arrangement as known in the art that is adapted to provide ultrashort-pulsed focused laser radiation, wherein at least a pulse length and a pulse energy or another energy-related parameter, such as a fluence per pulse, can be controlled. In view of the most relevant applications it is further preferable that the provided laser beam is a Gaussian beam with an M² parameter of no more than 1.15.

The control unit 530 may store and process data that is representative of a relationship between a pulse duration and a damage threshold pulse energy or a damage threshold for another energy-related pulse parameter, such as a threshold fluence, according to the present invention. For that purpose of data storage, the control unit 530 as shown in FIG. 5 comprises a storage device 535 that serves to host a data base. In alternative embodiments, the data base 535 may be arranged external to the control unit 530, provided that a functional connection between the control unit 530 and the data base 535 allows the control unit 530 to read and process data that is stored by the data base 535. Based on that stored data, the control unit 530 is configured to determine for a given pulse duration shorter than 200 fs an associated threshold pulse energy and, further, to determine an energy for pulses in a target beam based on the determined threshold value.

As shown in FIG. 5, the control unit 530 is further configured to output to the output device 540 a signal that indicates the determined target pulse energy. Graphical display of the determined target pulse energy thus enables a user of the laser apparatus to set a pulse energy according to the displayed information and dependent on the chosen pulse length. Alternatively, the control unit 530 may be adapted to set the determined pulse energy automatically. This can be achieved, for example, by control connections between the control unit and the beam source 510 and/or the set 520 of beam guiding and shaping means. This will allow a user of the laser apparatus 500 to arbitrarily vary the pulse length, whereas the laser apparatus 500 will automatically provide a corresponding pulse energy.

1. A method for energy setting of pulsed, focused laser radiation, the method comprising:
establishing a relationship between a threshold pulse energy and a pulse duration, the threshold pulse energy being the pulse energy required for causing irreversible damage in a material, the relationship allowing to obtain a threshold pulse energy for each of a plurality of pulse durations, the plurality of pulse durations including one or more pulse durations in a range between 200 fs and smaller, the relationship defining a decreasing threshold pulse energy for a decreasing pulse duration in the range between 200 fs and smaller;
for a given pulse duration in the range between 200 fs and smaller, determining an associated threshold pulse energy based on the established relationship; and
setting the pulse energy of the laser radiation based on the determined associated threshold pulse energy.

2. The method of claim 1, wherein the relationship represents a decrease of the threshold pulse energy substantially as a function of the cubic root of the pulse duration.

3. The method of claim 1, wherein the relationship defines the threshold pulse energy as a value of at most 0.35 μJ for a pulse duration of 300 fs or smaller.

4. The method of claim 1, wherein the relationship defines the threshold pulse energy as a value in the range from 0.15 μJ to 0.30 μJ for a pulse duration of 200 fs.

5. The method of claim 1, wherein the relationship defines the threshold pulse energy as a value in the range from 0.05 μJ to 0.10 μJ for a pulse duration of 10 fs.

6. The method of claim 1, wherein the establishing step includes:
irradiating, for each of a plurality of reference pulse durations above 200 fs, an object with a series of pulses of the laser radiation to create a damage site for each pulse of the series, wherein the pulse energy is set differently for each pulse of the series;
determining a size of each damage site;
determining a reference threshold pulse energy for each of the plurality of reference pulse durations based on the determined sizes of the damage sites created at the respective reference pulse duration; and
determining the relationship based on the determined reference threshold pulse energies.

7. The method of claim 6, wherein each reference threshold pulse energy is determined based on an extrapolation to zero size of the determined sizes of the damage sites created at the respective reference pulse duration.

8. The method of claim 6, wherein determining the relationship includes determining a linear approximation of the threshold pulse energy in dependence on the pulse duration.

9. The method of claim 1, wherein:
the relationship is established between the pulse duration and, in place of the threshold pulse energy, a threshold pulse fluence required for causing irreversible damage in the material,
the relationship defines a decreasing threshold pulse fluence for a decreasing pulse duration in the range between 200 fs and smaller, and
an associated threshold pulse fluence is determined in place of the associated threshold pulse energy and the pulse fluence of the laser radiation is set based on the determined associated threshold pulse fluence.

10. The method of claim 9, wherein the relationship defines the threshold pulse fluence as a value of at most 1.80 Jcm-2 for a pulse duration of 300 fs or smaller.

11. The method of claim 9, wherein the relationship defines the threshold pulse fluence as a value in the range from 0.80 Jcm-2 to 1.50 Jcm-2 for a pulse duration of 200 fs.

12. The method of claim 9, wherein the relationship defines the threshold pulse fluence as a value in the range from 0.20 Jcm-2 to 0.50 Jcm-2 for a pulse duration of 10 fs.

13. The method of claim 1, wherein the relationship is established for a focus diameter of the laser radiation of no more than 10 μm, wherein the focus diameter represents the diameter of a pulse portion containing 86% of the energy of a pulse of the radiation.

14. The method of claim 1, wherein the damage includes a photodisruption caused by a laser-induced optical breakdown of the material.

15. The method of claim 6, wherein the object is a non-biological material or a post mortem biological material.

16. (canceled)

17. (canceled)

18. A laser apparatus comprising:
a source of a beam of ultrashort-pulsed laser radiation;
a set of components that can guide and shape the beam in time and space;
a control unit storing data representative of a relationship between a threshold pulse energy required for causing irreversible damage in a material and a pulse duration, the relationship allowing to obtain a threshold pulse energy for each of a plurality of pulse durations, the plurality of pulse durations including one or more pulse durations in a range between 200 fs and smaller, the relationship defining a decreasing threshold pulse energy for a decreasing pulse duration in the range between 200 fs and smaller, wherein the control unit is configured to:
determine for a given pulse duration in the range between 200 fs and smaller an associated threshold pulse energy based on the stored data; and
determine a target pulse energy for the beam based on the determined associated threshold pulse energy.

19. The laser apparatus of claim 18, wherein the control unit is configured to output a visual representation of the determined target pulse energy on an output device.

20. The laser apparatus of claim 18, wherein the control unit is configured to set the determined target pulse energy for the beam automatically.

21. The laser apparatus of claim 18, wherein the relationship represents a decrease of the threshold pulse energy substantially as a function of the cubic root of the pulse duration.

22. The laser apparatus of claim 18, wherein the relationship defines the threshold pulse energy as a value of at most 0.35 μJ for a pulse duration of 300 fs or smaller.

23. The laser apparatus of claim 18, wherein the relationship defines the threshold pulse energy as a value in the range from 0.15 μJ to 0.30 μJ for a pulse duration of 200 fs.

24. The laser apparatus of claim 18, wherein the relationship defines the threshold pulse energy as a value in the range from 0.05 μJ to 0.1 μJ for a pulse duration of 10 fs.

25. The laser apparatus of claim 18, wherein the beam is a Gaussian beam having a M2 parameter of no more than 1.15 or 1.1.
